(19) United States
(12) Patent Application Publication
(10) Pub. No.: US 2022/0298329 A1
(43) Pub. Date: Sep. 22, 2022

(54) SEMI-SOLID STATE NUCLEIC ACID MANIPULATION

(71) Applicant: Keygene N.V., Wageningen (NL)

(72) Inventors: Raymond Jozef Maurinus HULZINK, Wageningen (NL); Stefan John WHITE, Wageningen (NL)

(21) Appl. No.: 17/837,904

(22) Filed: Jun. 10, 2022

Related U.S. Application Data
(63) Continuation of application No. PCT/EP2020/085694, filed on Dec. 11, 2020.

(30) Foreign Application Priority Data
Dec. 12, 2019 (EP) 19215708.9

(51) Int. Cl. C08L 5/04; C12Q 1/6806; C12N 15/10

(52) U.S. Cl. CPC C08L 5/04; C12Q 1/6806; C12N 15/1065

(57) ABSTRACT

The invention pertains to a method for isolating a nucleic acid, wherein the nucleic acid is stabilized in a hydrogel. The hydrogel can be dissolved to release the nucleic acid without breaking the molecule. A preferred hydrogel is alginate. The invention further concerns a method for sequencing the nucleic acid and a composition comprising the hydrogel and the nucleic acid.

### CROSS-REFERENCE TO RELATED APPLICATIONS

The present application is a continuation application of PCT/EP2020/085694 filed Dec. 11, 2020, which claims priority to EP 19215708.9 filed Dec. 12, 2019 the entire contents of both which are incorporated herein by reference.

### FIELD OF THE INVENTION

The present invention is in the field of molecular biology, more particularly in the field of genomics. In particular, the invention is in the field of sequencing, preferably the production and sequencing of long read sequencing libraries.

### BACKGROUND

The ability to obtain ultra-high molecular weight (uHMW) DNA and long read sequencing libraries is becoming increasingly important, especially for those applications where long-range genomic and genetic information is essential (e.g. whole genome mapping and sequencing). In particular, it has become evident that long sequence reads significantly improve the quality of de novo genome assemblies, especially for those species harbouring large and/or complex genomes. Moreover, long-read sequencing technologies hold the potential to resolve long repeats, polyploidy, and haplotypes and facilitate the identification of genetic elements associated with complex traits through CNV detection.

Nowadays, long-read sequencing technologies are able to produce reads of hundreds of kilobases in size with the potential to go even beyond megabase-sized read lengths. Consequently, the length of the DNA molecules primarily limits the read length of (e.g. nanopore-based) long-read sequencing technologies. As current library preparation protocols require dissolved genomic DNA as input, DNA isolation is still necessary. However, genomic DNA is highly prone to breakage upon handling in aqueous solutions and, as a consequence, using current state-of-the-art DNA isolation and library preparation protocols, long-read sequencing hardly yield sequences larger than ˜100 kb in size.

In the last few years, a number of initiatives have been undertaken worldwide to address challenges related to isolation of high quality, uHMW DNA and the generation of (ultra) long sequence reads. A few of these initiatives already resulted in commercialized technologies and other technologies are currently under development or being prepared for commercialization. Examples are uHMW DNA isolation and purification systems of Boreal Genomics and Sage Science, uHMW DNA isolation and purification kits of Circulomics and Evrogen, and automated DNA isolation and library preparation systems of Oxford Nanopore Technologies (i.e. Voltrax). Despite all of these developments, long-read sequencing currently hardly yield sequences larger than ˜100 kb in size and the prospect to sequence statistic values dominated by megabase-sized reads is still far away. Currently available sequencing methods mainly rely on isolation and/or manipulation (among others, library synthesis) of DNA molecules dissolved in aqueous solutions. As indicated above, dissolved DNA is highly prone to shearing upon handling due to physico-chemical forces, preventing the synthesis of sequencing libraries containing ultra-long megabase-sized DNA molecules.

It has previously been suggested in the art to use agarose hydrogels for capturing genomic DNA molecules (see e.g. Zhang et al, “Preparation of megabase-sized DNA from a variety of organisms using the nuclei method for advanced genomics research” (2012), Nature protocols, vol. 7 (3):467-478). However, extracting the (ultra) long DNA molecules from the hydrogel is known to result in shearing of the long nucleic acid molecules.

This shearing of DNA is also a problem when rinsing the long DNA molecules, e.g. after isolating the DNA from a cellular environment or after performing an enzymatic reaction.

Hence, there is still a need in the art to prevent the breaking or shearing of (ultra) high molecular weight nucleic acid molecules, e.g. when purifying or isolating the nucleic acids from cells and organelles molecules. Moreover, there is a need in the art for a method for sequencing the ultra-long nucleic acid molecules.

### SUMMARY

The invention may be summarized in the following numbered embodiments:

Embodiment 1. A method for obtaining a hydrogel comprising a manipulated nucleic acid, wherein the method comprises the steps of:
a) combining a nucleic acid with an aqueous polymer solution;
b) gelling the polymer solution to form a hydrogel comprising the nucleic acid; and
c) manipulating the nucleic acid in the hydrogel,
wherein the hydrogel can be dissolved at a temperature below 45° C.

Embodiment 2. A method according to embodiment 1, wherein the nucleic acid is comprised in a carrier and wherein preferably the carrier is at least one of an organelle and a cell and wherein the at least one of an organelle and a cell is lysed in step c to release the nucleic acid.

Embodiment 3. A method according to embodiment 1 or 2, wherein the polymers in the aqueous solution are ionic polymers, preferably anionic polymers having carboxylic pendant groups.

Embodiment 4. A method according to any one of the preceding embodiments, wherein the polymers in the aqueous solution are polysaccharides or derivatives thereof, preferably wherein said polysaccharides or derivatives thereof comprise an uronic acid.

Embodiment 5. A method according to embodiment 4, wherein the polysaccharide or the derivative thereof is alginate or a derivative thereof, preferably wherein the polysaccharide or the derivative thereof is alginate.

Embodiment 6. A method according to any one of embodiments 2-5, wherein the carrier is a mitochondrion, a chloroplast or a nucleus, wherein preferably the carrier is a nucleus.

Embodiment 7. A method according to any one of the preceding embodiments, wherein the nucleic acid is a DNA molecule, preferably a genomic DNA molecule.

Embodiment 8. A method according to any one of the preceding embodiments, wherein the manipulated nucleic acid is an isolated ultra-high molecular weight (uHMVV) nucleic acid.

Embodiment 9. A method according to any one of the preceding embodiments, wherein the cell is a plant cell, wherein preferably the plant cell is a protoplast.

Embodiment 10. A method according to any one of the preceding embodiments, wherein the manipulated nucleic acid is stabilized in a hydrogel microsphere.

Embodiment 11. A method for preparing a sequencing library, preferably a long-read sequencing library, comprising the steps of
obtaining the hydrogel comprising an manipulated nucleic acid as defined in any one of embodiments 1-10; and
modifying the nucleic acid in the hydrogel to obtain a sequencing library.

Embodiment 12. A sequencing method, preferably a deep-sequencing method, more preferably a long-read deep-sequencing method, comprising the steps of:
obtaining a sequencing library as defined in embodiment 11;
dissolving the hydrogel, preferably at a temperature below 45° C.; and
sequencing the library.

Embodiment 13. A method according to embodiment 12, wherein the sequencing library is loaded on a sequencer flow cell before dissolving the hydrogel, wherein preferably the flow cell is a flow cell of a long-read sequencer.

Embodiment 14. A method according to embodiment 13, wherein the sequencer is a nanopore sequencer.

Embodiment 15. A method according to any one of embodiments 12-14, wherein the hydrogel is dissolved by at least one of :
the addition of a sequencing buffer;
the addition of a buffer comprising monovalent cations, preferably sodium cations;
lowering the temperature from about 20° C.-40° C. to about 2° C.-10° C.; and
adjusting the pH from about 5-6 to about 7-8, or from about 7-8 to about 5-6.

Embodiment 16. A method for obtaining a hydrogel comprising a nucleic acid-comprising carrier, wherein the method comprises the steps of:
combining the nucleic acid-comprising carrier with an aqueous polymer solution; and
gelling the polymer solution to form the hydrogel comprising the nucleic acid-comprising carrier,
   wherein the hydrogel is a hydrogel as defined in any one of embodiments 1-5.

Embodiment 17. A method according to embodiment 16, wherein the nucleic acid-comprising carrier is an organelle and wherein the method further comprises a step of lysing a cell to obtain the organelle.

Embodiment 18. A hydrogel obtainable by the method of any one of embodiments 1-10, 16 and 17.

Embodiment 19. A hydrogel comprising at least one of a nucleic acid-comprising carrier, an organelle and a manipulated nucleic acid, preferably an isolated uHMW nucleic acid, wherein the hydrogel is a hydrogel as defined in any one of embodiments 1-5.

Embodiment 20. A hydrogel according to embodiment 19, wherein the hydrogel comprises alginate.

Embodiment 21. Use of a hydrogel as defined in any one of embodiments 18-20 for at least one of
genome sequencing;
long-range genome analysis;
transcriptome analysis;
map-based cloning;
genome physical mapping;
the construction of a large-insert BAC library; and
the construction of a large insert BIBAC library.

Embodiment 22. Kit of parts for obtaining a hydrogel comprising a manipulated nucleic acid, wherein the kit comprises:
i) a polymer for forming a hydrogel as defined in any one of embodiments 1-5;
ii) a cell and/or organelle lysis buffer; and
iii) optionally, one or more components for preparing a sequencing library.

Embodiment 23. A kit of parts according to embodiment 22, wherein the components for preparing a sequencing library comprise at least one or more adapters.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N.Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: the term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

As used herein, the term “adapter” is a single-stranded, double-stranded, partly double-stranded, Y-shaped or hairpin nucleic acid molecule that can be attached, preferably ligated, to the end of other nucleic acids, e.g., to one or both strands of a double-stranded DNA molecule, and preferably has a limited length, e.g., about 10 to about 200, or about 10 to about 100 bases, or about 10 to about 80, or about 10 to about 50, or about 10 to about 30 base pairs in length, and is preferably chemically synthesized. The double-stranded structure of the adapter may be formed by two distinct oligonucleotide molecules that are base paired with one another, or by a hairpin structure of a single oligonucleotide strand. As would be apparent, the attachable end of an adapter may be designed to be compatible with, and optionally ligatable to, overhangs made by cleavage by a restriction enzyme and/or programmable nuclease, may be designed to be compatible with an overhang created after addition of a non-template elongation reaction (e.g., 3′-A addition), or may have blunt ends.

“Amplification” used in reference to a nucleic acid or nucleic acid reactions, refers to in vitro methods of making copies of a particular nucleic acid, such as a target nucleic acid, or a tagged nucleic acid. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that is hybridizable, but not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Construct” or “nucleic acid construct” or “vector”: this refers to a man-made nucleic acid molecule resulting from the use of recombinant DNA technology and which can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g., a coding sequence) is integrated downstream of the transcription regulatory sequence. Vectors may comprise further genetic elements to facilitate their use in molecular cloning, such as e.g., selectable markers, multiple cloning sites and the like.

The terms “double-stranded” and “duplex” as used herein, describes two complementary polynucleotides that are base-paired, i.e., hybridized together. Complementary nucleotide strands are also known in the art as reverse-complement.

The term “effective amount,” as used herein, refers to an amount of a biologically active agent that is sufficient to elicit a desired biological effect. For example, in some embodiments, an effective amount of an exonuclease may refer to the amount of the exonuclease that is sufficient to induce cleavage of an unprotected nucleic acid. As will be appreciated by the skilled artisan, the effective amount of an agent may vary depending on various factors such as the agent being used, the conditions wherein the agent is used, and the desired biological effect, e.g. degree of nuclease cleavage to be detected.

“Exemplary”: this terms means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn can be translated into a protein or peptide.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “nucleotide” includes, but is not limited to, naturally-occurring nucleotides, including guanine, cytosine, adenine and thymine (G, C, A and T, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial or chloroplast DNA, RNA, such as mRNA, DNA from a library and/or RNA from a library. The nucleic acid for use in the invention may be isolated from e.g. a cell, tissue, biopsy or bodily fluid.

The nucleic acid used in the method of the invention can be from any source, e.g., a whole genome, a collection of chromosomes, a single chromosome, one or more regions from one or more chromosomes or transcribed genes, and may be isolated directly from the biological source or from a laboratory source, e.g., a nucleic acid library.

A nucleic acid for use in the method of the invention can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotides such as methylated DNA, and mimetics and combinations thereof.

The term “sequence of interest”, “target nucleotide sequence of interest” and “target sequence” are used interchangeably herein and includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said nucleic acid sequence of interest may be present in a double or a single strand nucleic acid. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. The sequence of interest can be, but is not limited to, a sequence having or suspected of having, a polymorphism, e.g. a SNP.

“Plant” refers to either the whole plant or to parts of a plant, such as cells, protoplasts, calli, tissue, organs (e.g. embryos pollen, ovules, seeds, gametes, roots, leaves, flowers, flower buds, anthers, fruit, etc.) obtainable from the plant, as well as derivatives of any of these and progeny derived from such a plant by selfing or crossing. Non-limiting examples of plants include crop plants and cultivated plants, such as African eggplant, alliums, artichoke, asparagus, barley, beet, bell pepper, bitter gourd, bladder cherry, bottle gourd, cabbage, canola, carrot, cassava, cauliflower, celery, chicory, common bean, corn salad, cotton, cucumber, eggplant, endive, fennel, gherkin, grape, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

An “endonuclease” is an enzyme that hydrolyses at least one strand of a duplex DNA or a strand of an RNA molecule, upon binding to its target or recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

An “exonuclease” is defined herein as any enzyme that cleaves one or more nucleotides from the end (exo) of a polynucleotide.

“Reducing complexity” or “complexity reduction” is to be understood herein as the reduction of a complex nucleic acid sample, such as samples derived from genomic DNA, isolated RNA samples and the like. Reduction of complexity results in the enrichment of one or more specific target sequences or target nucleic acid fragments (also denominated herein as target fragments) comprised within the complex starting material and/or the generation of a subset of the sample, wherein the subset comprises or consists of one or more specific target sequences or fragments comprised within the complex starting material, while non-target sequences or fragments are reduced in amount by at least 20%, 30%, 40%, 50%, 60%, 70%, 80%, 90%, 91%, 92%, 93%, 94%, 95%, 96%, 97%, 98% or 99% as compared to the amount of non-target sequences or fragments in the starting material, i.e. before complexity reduction. Reduction of complexity is in general performed prior to further analysis or method steps, such as amplification, barcoding, sequencing, determining epigenetic variation etc. Preferably complexity reduction is reproducible complexity reduction, which means that when the same sample is reduced in complexity using the same method, the same, or at least comparable, subset is obtained, as opposed to random complexity reduction. Examples of complexity reduction methods include for example AFLP® (Keygene N. V., the Netherlands; see e.g., EP 0 534 858), Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al ., 1999, The Plant Journal, vol. 20 (6) : 719-726) and modifications of SAGE (see e.g. Powell, 1998, Nucleic Acids Research, vol. 26 (14): 3445-3446; and Kenzelmann and Mühlemann, 1999, Nucleic Acids Research, vol. 27 (3): 917-918) , MicroSAGE (see e.g. Datson et al., 1999, Nucleic Acids Research, vol. 27 (5): 1300-1307), Massively Parallel Signature Seguencing (MPSS; see e.g. Brenner et al., 2000, Nature Biotechnology, vol. 18:630-634 and Brenner et al ., 2000, PNAS, vol. 97 (4):1665-1670) , self-subtracted cDNA libraries (Laveder et al., 2002, Nucleic Acids Research, vol. 30(9):e38), Real-Time Multiplex Ligation-dependent Probe Amplification (RT-MLPA; see e.g. Eldering et al., 2003, vol. 31 (23): e153), High Coverage Expression Profiling (HiCEP; see e.g. Fukumura et al., 2003, Nucleic Acids Research, vol. 31(16) :e94), a universal micro-array system as disclosed in Roth et al.(Roth et al., 2004, Nature Biotechnology, vol. 22 (4): 418-426), a transcriptome subtraction method (see e.g. Li et al., Nucleic Acids Research, vol. 33 (16): el36) , and fragment display (see e.g. Metsis et al., 2004, Nucleic Acids Research, vol. 32 (16): el27).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained. The term “next-generation sequencing” refers to the so-called parallelized sequencing-by-synthesis or sequencing-by-ligation platforms, e.g., such as currently employed by Illumina, Life Technologies, PacBio and Roche etc. Next-generation sequencing methods may also include nanopore sequencing methods, such as those commercialized by Oxford Nanopore Technologies, or electronic-detection based methods such as Ion Torrent technology commercialized by Life Technologies.

### DETAILED DESCRIPTION OF THE INVENTION

The inventors discovered a novel method for obtaining a manipulated nucleic acid molecule, preferably isolated from a cell and/or an organelle, while minimizing shearing of the nucleic acid molecule and increasing efficiency of a possible enzymatic manipulation step. The method as detailed herein avoids the need to isolate and manipulate DNA in an aqueous solution. Exemplary embodiments of the invention are shown in FIG. 1. The invention is based on the stabilisation of (long) nucleic acids in a hydrogel particles (exemplified in e.g. FIG. 1B). In one embodiment (exemplified in e.g. FIG. 1A), the invention concerns the extraction, and optionally the manipulation (for example, library preparation), of nucleic acids from tissues, cells or organelles that are encapsulated in cross-linked hydrogel particles. After lysis of the tissues, cells or organelles, the free uHMW DNA will become trapped in the hydrogel matrix and becomes accessible for (enzymatic) manipulation such as sequence library preparation. Finally, the hydrogel containing the manipulated DNA (e.g. sequence library) can be processed further (e.g. loading in a nanopore flow cell). Since all steps are carried out on physically immobilized DNA, the impact of damage on DNA (or library) is much lower compared to the traditional in-solution-based methods. Furthermore, immobilization of DNA allows a more efficient purification of the molecules from contaminants and enzymes. The invention is however not limited to biological carriers. As exemplified in FIG. 1C, non-biological carriers stabilising and/or precipitating the long nucleic acid molecules can likewise become encapsulated. The released nucleic acid molecules may subsequently be purified and/or manipulated in the hydrogel of the invention.

Therefore in a first aspect, the invention concerns a method for obtaining a hydrogel comprising a manipulated nucleic acid molecule, wherein said method comprises the steps of:
a) combining a nucleic acid with an aqueous polymer solution;
b) gelling the polymer solution to form the hydrogel comprising the nucleic acid; and
c) manipulating the nucleic acid in the hydrogel.

The “manipulating” of step c is to be understood herein as at least one of washing, isolating or releasing, enzymatically modifying (such as, but not limited to degrading, fragmenting, introducing a double-strand break or single strand break and deaminating), tagging, adapter ligation, elongating, end repairing, creating blunt ends, amplifying and any combination thereof. The hydrogel is a preferably a hydrogel as defined herein.

Preferably, the nucleic acid is comprised in or attached to a carrier, which is indicated herein as a carrier comprising a nucleic acid of nucleic acid-comprising carrier. Therefore, in an embodiment, the invention provides for a method for obtaining a hydrogel comprising a manipulated nucleic acid molecule, wherein said method comprises the steps of:
a) combining a nucleic acid-comprising carrier with an aqueous polymer solution;
b) gelling the polymer solution to form the hydrogel comprising the nucleic acid-comprising carrier; and
c) manipulating the nucleic acid of the nucleic acid-comprising carrier in the hydrogel.

The hydrogel is a preferably a hydrogel as defined herein. The nucleic acid-comprising carrier can be a natural or a non-natural nucleic acid-comprising carrier, i.e. a carrier that occurs in nature or a man-made nucleic acid-comprising carrier, as further detailed herein. Possible natural nucleic acid comprising carriers are cells and/or organelles.

In case the nucleic acid-comprising carrier is a cell, the method may further comprise a step of lysing a cell to obtain the organelle. The cell may be lysed before combining the organelle with an aqueous polymer solution. Alternatively, the cell may be lysed after combining the cell with an aqueous polymer solution and before gelling the polymer solution. Alternatively, the cell may be lysed after gelling the polymer solution to form a hydrogel comprising the organelle. Preferably, the hydrogel comprising an organelle does not, or does not substantially, comprise intact cells.

In case the carrier is a cell, the manipulating step c is preferably a step releasing the nucleic acid from the carrier, thereby obtaining a hydrogel comprising an isolated nucleic acid. The invention therefore also pertains to a method for obtaining a hydrogel comprising an isolated nucleic acid. The terms “isolated” and “extracted” can be used interchangeably herein and refers to the release from a nucleic acid from the carrier, optionally from its subcellular location. Therefore, the invention also provides for a method for obtaining a hydrogel comprising an isolated nucleic acid, wherein the method comprises the steps of:
a) combining a carrier comprising the nucleic acid with an aqueous polymer solution;
b) gelling the polymer solution to form a hydrogel comprising the carrier; and
c) releasing the nucleic acid from the carrier to obtain a hydrogel comprising the isolated nucleic acid.
   Preferably, the hydrogel can be dissolved at a temperature below 45° C.

In an embodiment, the carrier is at least one of an organelle and a cell. The nucleic acid is preferably isolated from the cell or the organelle. The at least one of an organelle and cell may be lysed to release the nucleic acid. Hence in this embodiment, the method preferably comprises the steps of:
a) combining at least one of an organelle and a cell comprising the nucleic acid with an aqueous polymer solution;
b) gelling the polymer solution to form a hydrogel comprising at least one of the organelle and the cell; and
c) lysing the at least one of an organelle and a cell to obtain the hydrogel comprising the isolated nucleic acid.

Preferably, the nucleic acid is stabilized in the hydrogel. The terms “stabilized” and “immobilized” can be used interchangeably herein. Stabilization of the nucleic acid in the hydrogel prevents breakage or shearing of the nucleic acid.

The nucleic acid-comprising carrier, preferably the at least one of an organelle and cell, may first be combined with the aqueous polymer solution, followed by a step of gelling the polymer solution.

Alternatively or in addition, the nucleic acid-comprising carrier, preferably the at least one of an organelle and a cell may be combined during the gelling of the aqueous polymer solution.

Lysing at least one of the cell and organelle releases the nucleic acid into the hydrogel. Hence, the method of the invention further concerns a method for obtaining an isolated nucleic acid, wherein the method comprises a step of lysing at least one of an organelle and a cell in a hydrogel to obtain an isolated nucleic acid, wherein the isolated nucleic acid is stabilized in the hydrogel, preferably a hydrogel as defined herein below.

Optionally, in addition to the steps a to c described above, the method of the invention may further comprise a step of:
d) dissolving the hydrogel comprising the (isolated) nucleic acid and/or nucleic acid-comprising carrier.

### Nucleic Acid

The nucleic acid obtainable by the method of the invention preferably has a size of at least about 10 kb (kilobases), 20 kb, 30 kb, 40 kb, 50 kb, 60 kb, 70 kb, 80 kb, 90 kb, 100 kb, 150 kb, 200 kb, 300 kb, 400 kb, 500 kb, 600 kb, 700 kb, 800 kb, 900 kb or at least about 1000 kb (1 Mb). Preferably, at least about 400 kb, 500 kb, 600 kb, 700 kb, 800 kb, 900 kb or at least about 1000 kb (1 Mb).

The nucleic acid is a nucleic acid obtainable by the method of the invention. The size of the nucleic acid may therefore understood herein as being the size of the (long) manipulated nucleic acid as defined herein.

Preferably, the method of the invention as detailed herein can be used for obtaining long, long-range, or high molecular weight (HMW) nucleic acids, i.e. having a size as indicated above. Preferably, the method of the invention as detailed herein can be used for obtaining ultra-high molecular weight (uHMW) nucleic acids. uHMW nucleic acids are preferably a subset of the long nucleic acids and may have a length of at least 1 Mb. Preferably, the nucleic acid obtainable by the method of the invention has a size of at least 1.1 Mb, 1.3 Mb, 1.5 Mb, 1.7 Mb, 2 Mb, 2.5 Mb, 3 Mb, 4 Mb, 5 Mb, 6 Mb, 7 Mb, 8 Mb, 9 Mb or at least about 10 Mb (megabases).

The nucleic acid obtainable by the method of the invention may be, or may be obtainable from, a nuclear genome or a cytoplast organellar genome. The nucleic acid molecule may be a naturally occurring nucleic acid or an artificial nucleic acid. The nucleic acid may be DNA, RNA or a mixture thereof. The nucleic acid can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotide such as methylated DNA, and mimetics and combinations thereof. The nucleic acid can be a partly or a fully single-stranded, double-stranded or triple-stranded molecule.

The nucleic acid obtainable by the method of the invention may be derived from any source, e.g. human, animal, plant, microorganism, and may be of any kind, e.g. endogenous or exogenous to the cell, for example genomic DNA, chromosomal DNA, artificial chromosomes, plasmid DNA, or episomal DNA, cDNA, RNA, mitochondrial DNA, chloroplast DNA, or of an artificial library such as a BAC or YAC or the like. The DNA may be nuclear or organellar DNA. Preferably, the DNA is genomic DNA, preferably endogenous to the cell.

The nucleic acid obtained in the method of the invention may comprise a sequence of interest, preferably a sequence of interest as defined herein above.

Preferably, the nucleic acid molecule is a DNA molecule, preferably a genomic DNA molecule.

### Nucleic Acid-Comprising Carrier

Preferably, the nucleic acid is isolated from a nucleic acid-comprising carrier. The nucleic acid comprising carrier may be a DNA-carrier. The nucleic acid-comprising carrier can be a natural or non-natural nucleic acid-comprising carrier, i.e. a carrier that occurs in nature or a man-made nucleic acid-comprising carrier.

A non-limiting example of a non-natural nucleic acid-comprising carrier is a carrier comprising one or more isolated chromosomes. The isolated chromosomes may be precipitated chromosomes, e.g. with or without additional compounds or chemical treatment to maintain the chromosomes in pelleted form. A non-natural nucleic acid comprising carrier may be solid or semi-solid, comprising nucleic acids or to which nucleic acids are attached.

Preferably, the nucleic acid-comprising carrier is a natural nucleic acid-comprising carrier. The natural nucleic acid-comprising carrier is preferably at least one of a cell and an organelle. Hence preferably, the nucleic acid is isolated from at least one of a cell and an organelle. The organelle is preferably contained within a cell and comprises a nucleic acid, preferably a nucleic acid as defined herein. An organelle for use in the method of the invention may be defined herein can be a membrane-bound or non-membrane bound organelle.

The organelle may be a membrane-bound organelle, e.g. the organelle comprises a lipid bilayer, preferably a phospholipid bilayer. The lipid bilayer, preferably the phospholipid bilayer, surrounds the nucleic acid. Hence a preferred organelle comprises a nucleic acid, preferably a nucleic acid as defined herein. A preferred membrane-bound organelle is at least one of a nucleus, a chloroplast, a mitochondrion, an endoplasmic reticulum, a flagellum, a golgi apparatus and a vacuole. A preferred organelle is at least one of a nucleus, a chloroplast and a mitochondrion. A preferred membrane-bound organelle is a nucleus. A preferred organelle for use in the method of the invention is a nucleus.

The organelle for use in the method of the invention may be a non-membrane bound organelle, i.e. not comprising a lipid bilayer. A preferred non-membrane bound organelle for use in the method of the invention is at least one of a nucleosome, ribosome, spliceosome, nucleolus, stress granule, a TIGER domain or a vault.

The nucleic acid for use in the method of the invention may be isolated from a cell, without additionally requiring isolating the nucleic acid from an organelle, in particular without requiring the isolation of the nucleic acid from a membrane-bound organelle. As a non-limiting example, the nucleic acid may be isolated from the cytoplasm of a cell. Such nucleic acid may for example constitute an RNA molecule or a prokaryotic DNA molecule.

The nucleic acid for use in the invention may be obtainable from any type of cell. The cell may be a viral particle, a prokaryotic cell or eukaryotic cell. The prokaryotic cell may be an archaeal cell or an bacterial cell. The cell may be a bacterial cell. A preferred bacterial cell may be at least one of Escherichia and Agrobacterium, preferably at least one of Escherichia coli and Agrobacterium tumefaciens.

The cell may be an eukaryotic cell selected from the group consisting of an animal cell, a plant cell and a fungal cell. The cell for use in the method of the invention may be an animal cell. The animal cell may be obtainable from the group consisting a rodent, a cat, a dog, cattle, a goat, a horse, a donkey, a sheep, a rabbit, a mice, a rat, a non-human primate and a human.

The cell for use in the method of the invention may be a plant cell. The plant cell may be a plant protoplast. The skilled person is aware of methods and protocols for preparing and propagating plant protoplasts, see for example Plant Tissue Culture (ISBN: 978-0-12-415920-4, Roberta H. Smith). The plant protoplasts for use in the method of the current invention can be provided using common procedures used for the generation of plant cell protoplasts, e.g. the cell wall may be degraded using cellulose, pectinase and/or xylanase prior to, or after, combining the cell with an aqueous polymer solution as defined herein.

Plant cell protoplasts systems have for example been described for tomato, tobacco and many more (Brassica napus, Daucus carota, Lactucca sativa, Zea mays, Nicotiana benthamiana, Petunia hybrida, Solanum tuberosum, Oryza sativa). The present invention is generally applicable to any protoplast system, including those, but not limited to, the systems described in any one of the following references: Barsby et al. 1986, Plant Cell Reports 5(2): 101-103; Fischer et al. 1992, Plant Cell Rep. 11(12): 632-636; Hu et al. 1999, Plant Cell, Tissue and Organ Culture 59: 189-196; Niedz et al. 1985, Plant Science 39: 199-204; Prioli and Söndahl, 1989, Nature Biotechnology 7: 589-594; S. Roest and Gilissen 1989, Acta Bot. Neerl. 38(1): 1-23; Shepard and Totten, 1975, Plant Physiol.55: 689-694; Shepard and Totten, 1977, Plant Physiol. 60: 313-316, which are incorporated herein by reference.

Preferably, the plant cell for use in the method of the invention may be a cell obtainable from a crop plant or a cultivated plant, i.e. plant species which is cultivated and bred by humans. A crop plant may be cultivated for food or feed purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork, fibres (such as cotton) and the like. Preferably, the cell as taught herein is from a crop plant.

The plant cell may be obtained from a monocot or dicot. A monocot plant may belong to the family of Poaceae. A dicot plant may be selected from the group consisting of Solanum, Capsicum, Nicotiana, Cucurbitaceae, Abelmoschus esculentus, Fabaceae, Asteraceae, Amaranthaceae, Brassicaceae, Lamiaceae and Rosaceae.

### Aqueous Polymer Solution

An aqueous polymer solution is defined herein as an aqueous solution comprising one or more polymers. Under specific conditions, an aqueous polymer solution, preferably in combination with a nucleic acid-comprising carrier, may form a hydrogel, which is defined herein as a three-dimensional, hydrophilic network embedded in an aqueous environment.

Gelling is defined herein as the formation of a hydrogel from an aqueous polymer solution, preferably in the presence of a nucleic acid-comprising carrier. Herein, gelation is used a synonym for gelling. During gelling crosslinks are formed between the polymers comprised in the aqueous polymer solution, and preferably also between said polymers and the nucleic acid-comprising carrier.

Said crosslinks may comprise covalent bonds, ionic bonds, molecular entanglements, hydrogen bonding, hydrophobic interactions, van der Waals forces and/or dipole-dipole interactions. Preferably, said crosslinks comprise covalent bonds and/or ionic bonds. The nature of these crosslinks is primarily determined by the chemical structure of the polymers and the nucleic acid-comprising carrier. For example, an aqueous solution comprising a polymer with ionic pendant groups may form ionic bonds upon gelling. A crosslink may comprise one or more linking compounds such as an ion.

Gelling may be a reversible process. Dissolution is defined as the reverse process of gelling. In other words, during dissolution an aqueous polymer solution, preferably in combination with one or more additional compounds, is formed from a hydrogel. Herein, dissolving is a synonym of dissolution. Notwithstanding the reversibility of gelling, the dissolution of a hydrogel, formed from a first aqueous solution, results in a second aqueous solution, wherein said first and second aqueous solution are not required to be identical.

It is understood herein that dissolution includes partly dissolving the hydrogel, e.g. dissolving at least about 1%, 5%, 10%, 15%, 20%, 25%, 30%, 35%, 40%, 45%, 50%, 55%, 60%, 65%, 70%, 75%, 80%, 85%, 90%, 95% or about 100% of the hydrogel.

Gelling and dissolution occur under specific conditions, which may be characterized by one or more parameters such as temperature, pH, the concentration of specific ions, and the presence of gelation or dissolution inducers, alone or in combination.

An aqueous polymer solution may have a lower critical gelation temperature (LCGT), around which gelling will occur upon heating. For example, an aqueous solution of Polaxamer or MeBiol has an LCGT. A hydrogel having an LCGT is defined herein as a hydrogel formed from an aqueous polymer solution having an LCGT. Reversibly, a hydrogel having an LCGT will dissolve upon cooling around the LCGT.

An aqueous polymer solution may have an upper critical gelation temperature (UCGT), around which gelling will occur upon cooling. For example, an aqueous solution of agarose has an UCGT. Therefore, a hydrogel formed from an aqueous solution of agarose and one or more organelles and/or cells has to be heated in order to dissolve. A hydrogel having an UCGT is defined as a hydrogel formed from an aqueous polymer solution having an UCGT. Reversibly, a hydrogel having an UCGT will dissolve upon heating around the UCGT.

Besides temperature, a specific condition for inducing gelling/dissolution may include the concentration of specific ions. For example, gelling an aqueous solution of alginate combined with one or more organelles occurs in the presence of calcium ions. Without being bound to this theory, the calcium ions allow the formation of ionic crosslinks between the carboxylic pendant groups of the alginate chains, as the ions act as linking compounds.

Moreover, a specific condition for inducing gelling or dissolution may include the presence of a gelation inducer or a dissolution inducer, respectively. A gelation inducer is a compound or a composition which has to be present with a minimal concentration in an aqueous polymer solution in order for gelling of said aqueous polymer solution to be able to occur. A dissolution agent is a compound or a composition which has to be present with a minimal concentration in a hydrogel on order for dissolution of said hydrogel to be able to occur.

Wherever an aqueous polymer solution which can be gelled below or above a given temperature is described herein, or wherever a hydrogel which can be dissolved below or above a given temperature is described herein, these temperature requirements for gelling or dissolution could be interpreted as necessary, but not always as sufficient conditions. For example, a hydrogel formed from an aqueous solution of alginate and one or more organelles and/or cells may be considered dissolvable at a temperature below 45° C., even if a significant lowering of the calcium concentration is required at said temperatures to induce the dissolution of said hydrogel.

Dissolution of a hydrogel may take place in the presence of a dissolution inducer. A dissolution inducer is an aqueous solution which is brought into contact or is combined with a hydrogel, preferably before the dissolution. Preferably, the introduction of a dissolution inducer helps to establish or induces the specific conditions needed for dissolution. For example, combining a dissolution inducer with a hydrogel formed from an aqueous polymer solution of alginate may lead to dissolution (or dissolving) of the hydrogel.

In the context of this invention, the term polymer is preferably used for a polymer comprised in an aqueous polymer solution which is able to form a hydrogel, preferably wherein said hydrogel may be dissolved under specific conditions.

Preferably, an aqueous polymer solution of the invention has an upper critical gelation temperate (UCGT) and/or may be gelled upon the increase or decrease of the concentration of one more specific ions comprised in the aqueous polymer solution. Correspondingly, a hydrogel preferably has an upper critical gelation temperate (UCGT) and/or may be dissolved upon the increase or decrease of the concentration of one more specific ions comprised in the hydrogel.

Preferably, an aqueous polymer solution of the invention has an upper critical gelation temperature below about 60° C., 55° C., 50° C., 45° C., 40° C., 35° C., 30° C., 25° C., 20° C. Correspondingly, a hydrogel preferably has an upper critical gelation temperature below about 60° C., 55° C., 50° C., 45° C., 40° C., 35° C., 30° C., 25° C., 20° C.

Preferably, an aqueous polymer solution of the invention has a lower critical gelation temperate (LCGT) and/or may be gelled upon the increase or decrease of the concentration of one more specific ions comprised in the aqueous polymer solution. Correspondingly, a hydrogel preferably has a lower critical gelation temperate (LCGT) and/or may be dissolved upon the increase or decrease of the concentration of one more specific ions comprised in the hydrogel.

Preferably, an aqueous polymer solution of the invention has an lower critical gelation temperature above about 25° C., 30° C., 40° C., 45° C. Correspondingly, a hydrogel preferably has a lower critical gelation temperature below about 25° C., 30° C., 40° C., 45° C.

Preferably, an aqueous polymer solution and/or a hydrogel of the invention has a LCGT and/or a UCGT, preferably a LCGT or UCGT as defined herein.

Preferably, an aqueous polymer solution of the invention may be gelled upon the increase of the concentration of multivalent ions, preferably of multivalent cations, preferably of calcium. Said increase of the concentration is preferably at least an increase of about 10%, 20%, 30%, 40%, 50%, 100%, 200%, 300%, 400%, 500%, 1000% or more. Said increase of the concentration is preferably able to induce gelling at a temperature below about 60° C., 55° C., 50° C., 45° C., 40° C., 35° C., 30° C., 25° C., 20° C. Correspondingly, a hydrogel may be dissolved upon the decrease of the concentration of multivalent ions, preferably of multivalent cations, preferably of calcium. Said decrease of the concentration is preferably at least a decrease of about 10%, 20%, 30%, 40%, 50%, 100%, 200%, 300%, 400%, 500%, 1000% or more. Said decrease of the concentration is preferably able to induce dissolution at a temperature below about 60° C., 55° C., 50° C., 45° C., 40° C., 35° C., 30° C., 25° C., 20° C.

Preferably, an aqueous polymer solution of the invention may be gelled upon the decrease of the concentration of monovalent ions, preferably of monovalent cations, preferably of sodium or potassium, preferably of sodium. Said decrease of the concentration is preferably at least a decrease of about 10%, 20%, 30%, 40%, 50%, 100%, 200%, 300%, 400%, 500%, 1000% or more. Said decrease of the concentration is preferably able to induce gelling at a temperature below about 60° C., 55° C., 50° C., 45° C., 40° C., 35° C., 30° C., 25° C., 20° C. Correspondingly, a hydrogel may be dissolved upon the increase of the concentration of monovalent ions, preferably of monovalent cations, preferably of sodium or potassium, preferably of sodium. Said increase of the concentration is preferably at least an increase of about 10%, 20%, 30%, 40%, 50%, 100%, 200%, 300%, 400%, 500%, 1000% or more. Said increase of the concentration is preferably able to induce dissolution at a temperature below about 60° C., 55° C., 50° C., 45° C., 40° C., 35° C., 30° C., 25° C., 20° C.

Preferably, an aqueous polymer solution of the invention comprises an ionic polymer, preferably an anionic polymer, preferably an anionic polymer comprising carboxylic pendant groups. Correspondingly, a hydrogel is preferably formed from an aqueous polymer solution comprising an ionic polymer, preferably an ionic polymer, preferably an anionic polymer comprising carboxylic pendant groups, wherein said hydrogel comprises ionic crosslinks. Such ionic crosslinks comprise an ion, preferably calcium, as a linking compound.

In the context of this application, it is understood that an anionic compound or group refers to a compound or a group which is negatively charged under the conditions present in the corresponding aqueous polymer solution or hydrogel, notwithstanding that said compound or group may be neutral or positively charged under different conditions. A corresponding definition holds for neutral and cationic compounds and groups.

Preferably, an aqueous polymer solution of the invention comprises a polysaccharide or a derivative thereof. More preferably, said polysaccharide or derivative thereof comprises one or more sugar acids, preferably uronic acids. Preferably, an aqueous polymer solution of the invention comprises alginate or a derivative thereof. Correspondingly, a hydrogel is preferably formed from an aqueous solution comprising a polysaccharide or a derivative thereof, more preferably said polysaccharide or derivative thereof comprises one or more sugar acids, preferably uronic acids.

Alginate is the family of linear copolymers of (1,4)-linked β-D-mannuronate (M) and α-L-guluronate (G) residues or monomers. Said M and G monomers are arranged as consecutive G residues, consecutive M residues or alternating M and G residues in alginate. The ratio of the number of M and the number of G residues, and the lengths of the blocks of G residues, the blocks of M residues and the blocks of MG residues in an alginate is dependent on the source from which said alginate is obtained. A preferred source of alginate is from a species within the class of Phaeophycea (brown algea).The alginate may be obtainable from a species of at least one of Macrocystis, Sargassum and Laminaria. Preferably, the Laminaria is at least one of Laminaria digitate, Laminaria hyperborea and Laminaria Durvillaea.

Preferably, the G/M ratio is ≥1.5. Preferably, the M/G ratio is at least about 0.7 or 0.8, preferably about 0.7-1.4 or about 0.8-1.6.

The viscosity of the alginate when dissolved in water may be high, medium or low. Preferably the viscosity (mPa/s) is about 4-12 or about 20-200. Preferred alginate derivatives are amphiphilic alginates and alginates covalently attached to oligopeptides. Amphiphilic alginates are derivable from alginates by covalently attaching hydrophobic groups such as alkyls to the carboxylic pendant groups.

In the context of this application a polysaccharide also refers to a derivative of a polysaccharide, unless explicitly stated otherwise. In the context of this application an alginate also refers to a derivative of an alginate, unless explicitly stated otherwise.

Preferably, the nucleic acid is captured in the hydrogel and does not diffuse, or does not substantially diffuse, out of the hydrogel. Preferably, at least about 80%, 85%, 90%, 95%, 96%, 97%, 98%, 99% or 100% of the nucleic acid molecules remains in the hydrogel, when maintaining the hydrogel at room temperature for a period of at least about 60 minutes. Preferably, at least about 80%, 85%, 90%, 95%, 96%, 97%, 98%, 99% or 100% of the nucleic acid molecules remains in the hydrogel, when maintaining the hydrogel at room temperature for a period of at least about 8 hours.

Preferably, the hydrogel does not interfere, or does not substantially interfere, with any downstream processing. Preferably, the polymers of the hydrogel do not interfere, or do not substantially interfere, with sequencing, preferably deep-sequencing, the nucleic acid molecule.

### Combining the Organelle/Cell and Hydrogel

It is understood herein that the organelle and/or cell for use in the current invention preferably comprises a nucleic acid, preferably a nucleic acid as defined herein. In the method of the invention, the cell may first be lysed to release the organelles and the organelles are subsequently combined with the aqueous polymer solution as defined herein. The polymers can subsequently form (cross-)linked networks to obtain a hydrogel comprising the organelles.

Alternatively or in addition, an intact cell may be combined with the aqueous polymer solution and the organelle comprising the nucleic acid can be released from the cell prior to (cross-)linking the polymers to form a hydrogel.

Alternatively or in addition, an intact, or substantially intact, cell may be combined with the aqueous polymer solution and the organelle comprising the nucleic acid may be released from the cell after forming a hydrogel.

### Lysing the Organelle/Cell

An intact cell may be combined with the aqueous polymer solution and the polymers may subsequently be (cross-)linked to form a hydrogel. Lysing the cell in the hydrogel may isolate or release the nucleic acid without requiring the additional disruption of one or more organelles. For instance, the nucleic acid may be present in the cytoplasm of a cell, e.g. in the case of prokaryotic cells and/or the nucleic acid molecule may be an cytoplasmic RNA molecule. Hence depending on the subcellular location of the nucleic acid, the method may comprise a step of lysing an organelle comprising the nucleic acid.

Isolating the nucleic acid may require a step of lysing a cell and lysing an organelle. The term “lysing a cell” is understood herein as destroying, or breaking down, the cell membrane and optionally also breaking down the cell wall. Similarly, the term “lysing an organelle” is understood herein as destroying, or breaking down, the organellar membrane.

Cell lysis preferably results in the release of an organelle. Releasing an organelle from a cell can be achieved using any conventional method known in the art. Optionally, the cell is lysed prior to gelling. Alternatively, the cell is lysed after forming the hydrogel. Preferably, the organelle is lysed after forming the hydrogel.

The cell membrane, and optionally the cell wall, can be destroyed using any conventional method, such as, but not limiting to, mechanical force, enzymatic treatment, chemical treatment or osmotic treatment. Optionally, the organelle can be separated from at least one of the intact cells, cell debris and other type of organelles prior to combining the organelles comprising the nucleic acid with the aqueous polymer solution. Alternatively, the lysed cells are combined with the aqueous polymer solution as described herein. Cell lysis may also simultaneously result in lysis of the, nucleic acid-containing, organelle. Alternatively, there is first a step of cell lysis followed by a separate step of lysis of the organelle. Lysis of an organelle, preferably an organelle as defined herein, preferably results in the release of a nucleic acid, preferably a nucleic acid as defined herein. Lysis of an organelle can be achieved using any convention method known in the art, such as at least one of mechanical force, enzymatic treatment, chemical treatment and osmotic treatment. A non-limiting example of an enzymatic treatment is proteinase K treatment.

### Microsphere

The formed hydrogel may have any suitable size or shape. Preferably, the size of the hydrogel is suitable to dissolve the hydrogel at a temperature below 45° C. The size of the hydrogel is preferably suitable to dissolve the hydrogel in a manner and amount that is suitable to perform a subsequent step of at least one of genome sequencing, long-range genome analysis, transcriptome analysis, map-based cloning, genome physical mapping, the construction of a large-insert BAC library and the construction of a large insert BIBAC library. Preferably, the size of the hydrogel is suitable to dissolve the hydrogel under such conditions that make it feasible to perform a subsequent step of deep-sequencing, preferably long-read deep sequencing.

The size or shape of the hydrogel may be a particle. Preferably, the hydrogel can be a microparticle. Preferably, the hydrogel can be a microsphere. Preferably, the manipulated, preferably isolated, nucleic acid as defined herein is stabilized in a hydrogel microsphere. As used herein, the term “microsphere” refers to a microparticle that is substantially spherical in shape and is equal to or less than about 2 mm in diameter. For example, the microparticle may be substantially spherical in shape and is equal to or less than about 1 mm in diameter.

As used herein, “substantially spherical” generally means a shape that is close to a perfect sphere, which is defined as a volume that presents the lowest external surface area. For example, “substantially spherical” can refer to microspheres wherein, when viewing any cross-section of the microspheres, the difference between the major diameter (or maximum diameter) and the minor diameter (or minimum diameter) is less than about 20%, less than about 15%, less than about 10%, less than about 5%, or less than about 1%. The term “substantially spherical” can also refer to a microsphere having a major diameter/minor diameter ratio of from about 1.0 to about 2.0, from about 1.0 to about 1.5, or from about 1.0 to about 1.2.

Preferably, the microspheres are spherical or substantially spherical in shape. The diameter of the microspheres may vary. For example, in some embodiments, the microspheres have an average diameter of from about 10 μm to about 2,000 μm, from about 30 μm to about 1,500 μm, from about 35 μm to about 1,000 μm, from about 40 μm to about 900 μm, from about 45 μm to about 500 μm, or from about 20 μm to about 200 μm.

The microspheres can also be substantially uniform in size. For example, the difference in diameter between individual microspheres can be from about 0 μmm to about 250 μm, from about 0 μm to about 200 μm, from about 0 μm to about 150 μm, from about 0 μm to about 100 μm, or from about 0 μm to about 50 μm. In further embodiments, individual microspheres have differences in diameter of 200 μm or less, 150 μm or less, 100 μm or less, about 50 μm or less, about 25 μm or less, about 10 μm or less, or about 5 μm or less.

Preferably, the microspheres are in a population wherein greater than 50% have a diameter of ±20% of the mean, ±10% of the mean, or ±5% of the mean diameter. In one embodiment, the microspheres are in a population wherein greater than 75% have a diameter of ±20% of the mean, ±10% of the mean, or ±5% of the mean diameter.

Preferably the hydrogel microsphere comprising a nucleic acid as defined herein, preferably comprising a sequencing library as defined herein, is loaded onto a sequencer flow cell prior to dissolving the hydrogel and releasing the nucleic acid.

A hydrogel microsphere as defined herein may be produced using any conventional method known in the art. As a non-limiting example, the microsphere can be produced using microfluidic-based synthesis (e.g. as exemplified in FIG. 4), e.g. wherein the first reactant comprises at least one of the cell and organelle and the second reactant comprises the aqueous polymer solution. An optional third reactant may comprise a gelation inducer.

### Sequencing Library

The manipulated, preferably isolated, nucleic acid captured in the hydrogel can straightforwardly be modified and/or purified without fragmenting the nucleic acid. Surprisingly, performing an enzymatic reaction within the hydrogel significantly lowers the amount of nucleic acids required as input material as compared to performing the same reaction in an aqueous environment.

The components of e.g. an enzymatic reaction mixture may diffuse into the formed hydrogel matrix. Such components may include at least one of an enzyme, an adapter, an antibody, an oligonucleotide and a primer. A preferred enzyme for modifying the nucleic acid includes, but is not limited to, a specific DNA-targeting enzyme, a ligase, an endonuclease and an exonuclease. A preferred specific DNA targeting enzyme is a CRISPR nuclease complex.

In addition or alternatively, the enzymatic reaction may be a protein digestion, e.g. to remove proteins that are bound to the nucleic acid. A preferred enzymatic reaction is the addition of one or more proteases, such as, but not limited to a proteinase K.

The method may further comprise a step of inactivating the enzymes, e.g. the inactivation of a nucleic acid-modifying enzyme and/or inactivation of a proteinase.

In addition or alternatively, the nucleic acid may be purified by rinsing the hydrogel, preferably rinsing the hydrogel at least 1, 2, 3, 4, 5 or more times The nucleic acid, optionally the modified nucleic acid, may be purified e.g. to remove at least one of cellular debris, enzymes, unbound adapters and contaminants.

In an embodiment the organelle, preferably the nucleus, may be rinsed prior to releasing the nucleic acid from the organelle. Rinsing the organelle may e.g. remove cytoplasmic contaminants that otherwise bind the nucleic acid and which may disturb the sequencing process.

In an aspect, the manipulated nucleic acid molecule obtained by the method as defined herein above may be modified. In an embodiment, the invention pertains to a method for preparing a sequencing library. The sequencing library is preferably a long-read sequencing library. Preferably, the sequencing library is a deep-sequencing library. Preferably, the sequencing library may be used for next-generation and/or third generation sequencing. Preferably, the sequencing library may be used for long-read sequencing.

Preferably, the sequencing library comprises a nucleic acid as defined herein. Preferably, at least part of the nucleic acid molecules in the sequencing library have a size of at least about 10 kb, 20 kb, 30 kb, 40 kb, 50 kb, 100 kb, 150 kb, 200 kb, 300 kb, 400 kb, 500 kb, 600 kb, 700 kb, 800 kb, 900 kb or at least about 1000 kb (1 Mb). Preferably, at least part of the nucleic acid molecules in the sequencing library have a size of at least 1.1 Mb, 1.3 Mb, 1.5 Mb, 1.7 Mb, 2 Mb, 2.5 Mb, 3 Mb, 4 Mb, 5 Mb, 6 Mb, 7 Mb, 8 Mb, 9 Mb or at least about 10 Mb.

Preferably, the N50, or read N50, of the nucleic acid molecules in the sequencing library is at least about 10 kb, 20 kb, 30 kb, 40 kb, 50 kb, 100 kb, 150 kb, 200 kb, 300 kb, 400 kb, 500 kb, 600 kb, 700 kb, 800 kb, 900 kb or at least about 1000 kb (1 Mb). Preferably, the N50 of the nucleic acid molecules in the sequencing library have a size of at least 1.1 Mb, 1.3 Mb, 1.5 Mb, 1.7 Mb, 2 Mb, 2.5 Mb, 3 Mb, 4 Mb, 5 Mb, 6 Mb, 7 Mb, 8 Mb, 9 Mb or at least about 10 Mb. The N50 is defined herein as the value where half of the data is contained within reads with alignable lengths greater than this.

Preferably, at least part of the nucleic acid molecules of the sequencing library are high molecular weight (HMW) or ultra-high molecular weight (uHMW) nucleic acids.

Preferably the method of the invention for preparing a sequencing library comprises the steps of:
obtaining a hydrogel comprising the manipulated, preferably isolated, nucleic acid as defined herein; and
modifying the nucleic acid in the hydrogel to obtain a sequencing library.

To obtain a sequencing library, the nucleic acid may be modified while the molecule is maintained within the hydrogel. Stabilizing/immobilizing the nucleic acid in the hydrogel during the process of preparing a sequencing library may substantially reduce and/or prevent breakage of the long nucleic acid molecule as compared to preparing the sequencing library in a conventional aqueous solution.

The sequencing library can be prepared using any conventional methods known in the art for preparing a sequencing library. The reagents for preparing the sequencing library can access the nucleic acid while the nucleic acid remains immobilized in the hydrogel. Preferably, the sequencing library can be prepared by attaching one or more adapters to one or both ends of the nucleic acid. Preferably the one or more adapters are ligated to one or more ends of the nucleic acid molecule. The adapter or adapters may be single-stranded, double-stranded, partly double-stranded, Y-shaped, circularizable or hairpin adapters.

One or more adapters may be protective adapters. In this context, a protective adapter is to be understood herein as an adapter that is specifically designed to protect the nucleic acid captured by the adapter for exonuclease digestion. Such adapter preferably protects against exonuclease degradation either by the inclusion of chemical moieties or blocking groups (e.g. phosphorothioate) or by a lack of terminal nucleotides (hairpin or stem-loop adapters, or circularizable adapters).

These one or more adapters may comprise functional domains, preferably selected from the group consisting of a restriction site domain, a capture domain, a sequencing primer binding site, an amplification primer binding site, a detection domain, a barcode sequence, a transcription promoter domain and a PAM sequence, or any combination thereof. The barcode can be, but is not limited to, a sample barcode, or a unique molecular identifier (UMI).

Preferably, the one or more adapters are sequencing adapters, e.g. comprise a functional domain that allows for Roche 454A and 454B sequencing, ILLUMINA™ SOLEXA™ sequencing, Applied Biosystems' SOLID™ sequencing, the Pacific Biosciences' SMRT™ sequencing, Pollonator Polony sequencing, Oxford Nanopore Technologies or the Complete Genomics sequencing. Preferably, the functional domain allows for at least one of nanopore sequencing and single-molecule real-time (SMRT™) sequencing. Preferably, the functional domain allows nanopore sequencing.

Depending on the adapter design, the adapter or adapters may be a single-stranded, double-stranded, partly double-stranded, Y-shaped, hairpin or circularizable adapters. Optionally, one or more adapters may be used. Optionally, one or more sets of two adapters may be used, wherein a first adapter of a set is aimed to be ligated at the 5′ end side of the nucleic acid, and the second adapter of set is aimed to be ligated at the 3′ end side of the nucleic acid.

Preferably, the first and second adapter within a set each comprise compatible primer binding sequences, such that adapter ligated nucleic acids are ready to be either amplified using a compatible primer pair or sequenced.

Preferably, the sequencing library preparation method of the invention is free of amplification and/or cloning steps. Reduction of amplification steps is beneficial, as nucleotide modification information (e.g., 5-mC, 6-mA, etc.) will get lost in amplicons. Further amplification can introduce variation into the amplicons (e.g., via errors during amplification) such that their nucleotide sequence is not reflective of the original sample. Similarly, cloning of the nucleic acid into another organism often does not maintain modifications present in the original sample nucleic acid, so in preferred embodiments the sequencing library preparation method of the invention typically does not comprise an amplification or cloning step.

Stem-loop or hairpin adapters are single-stranded, but their termini are complementary such that the adapter folds back on itself to generate a double-stranded portion and a single-stranded loop. A stem-loop adapter can be linked to an end of a linear, double-stranded nucleic acid. For example, where stem-loop adapters are joined to the ends of a double-stranded nucleic acid, such that there are no terminal nucleotides (e.g., any gaps have been filled and ligated, using a polymerase and ligase, respectively), the resulting molecule lacks terminal nucleotides, instead bearing a single-stranded loop at each end.

The nucleic acid may be ligated to circularizable adapters. In this respect, the nucleic acid in the hydrogel may be circularized by self-circularization of compatible structures on either side of the fragment (which may result from adapter ligation or as a result of restriction enzyme digestion of ligated adapters) or circularized by hybridization to a selector probe that is complementary to the ends of the desired fragment. Extension and a final step of ligation creates a covalently closed circular, optionally double-stranded, nucleic acid.

Optionally, fragments of the nucleic acid may be modified to comprise an A-tail, preferably to facilitate ligation to a partly, or fully, double-stranded adapter comprising a T-overhang. Hence prior to annealing an adapter to the nucleic acid, the method of the invention may optionally comprise a step of A-tailing the nucleic acid. A-tailing reactions are well-known in the art and the skilled person straightforwardly understands how to perform an A-tailing reaction, such as e.g. using a Klenow fragment (exo-).

The method for preparing a sequencing library as defined herein may optionally include a step of complexity reduction. The skilled person straightforwardly understands how to perform a step of complexity reduction and the invention is not limited to any complexity reduction method step.

As a non-limiting example, the nucleic acid stabilized in the hydrogel may be digested by one or more endonucleases.

Enzymatic digestion for fragmenting the nucleic acid includes, but is not limited to, endonuclease restriction. Enzymatic digestion, such as e.g. used in AFLP® technology, may further result in a complexity reduction of the nucleic acid. The skilled person knows which enzymes to select for the DNA restriction. As a non-limiting example, at least one frequent cutter and at least one rare cutter can be used for the fragmentation of the nucleic sample. A frequent cutter preferably has a recognition site of about 3-5 bp, such as, but not limited to Msel. A rare cutter preferably has a recognition site of >5bp, such as but not limited to EcoRl.

In a preferred method, the endonuclease is a rare cutter.

The method of the invention is not limited to any specific restriction endonucleases. The endonuclease may be a type II endonuclease, such as EcoRl, Msel, Pstl etc. In certain embodiments a type IIS or type III endonuclease may be used, i.e. an endonuclease of which the recognition sequence is located distant from the restriction site, such as, but not limited to, AceIII, AIwI, AIwXI, AIw26I, BbvI, BbvII, BbsI, Bed, Bce83I, BcefI, BcgI, BinI, Bsa, BsgI, BsmAI, BsmFI, BspMI, EarI,EciI, Eco3II, Eco57I, Esp3I, FauI, FokI, GsuI, HgaI, HinGUII, HphI, Ksp632I, MboII, MmeI, MnII, NgoVIII, PIeI, RIeAI, SapI, SfaNI, TaqJI and ZthII III. Restriction fragments can be blunt-ended or have overhanging ends, depending on the endonuclease used.

The recognition site of at least one of the frequent cutter and the rare cutter is within or in close proximity of a sequence variant of interest, e.g. the recognition site of the frequent cutter or the rare cutter is located about 0-10000, 10-5000, 50-1000 or about 100-500 bases from the sequence variant of interest.

The current method as disclosed herein can also be used in AFLP® technology. The AFLP® technology is e.g. described in more detail in WO2007/114693, WO2006/137733 and WO2007/073165, which are incorporated herein by reference. The AFLP® technology as described in the art can be modified by attaching a UMI to the restricted nucleic acid sample.

In addition or alternatively, the nucleic acid may be digested using a programmable nuclease, preferably using at least one of CRISPR-Cas technology, Zinc finger nucleases, TALENs and meganucleases.

Enrichment, or complexity reduction, is defined herein above, and preferably the complexity reduction is reproducible complexity reduction. One or more complexity reduction steps can be used, such as, but not limited to, selected from the group consisting of Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al ., 1999, The Plant Journal, vol. 20 (6): 719-726) and modifications of SAGE (see e.g. Powell, 1998, Nucleic Acids Research, vol. 26 (14): 3445-3446; and Kenzelmann and Mühlemann, 1999, Nucleic Acids Research, vol. 27 (3): 917-918), MicroSAGE (see e.g. Datson et al., 1999, Nucleic Acids Research, vol. 27 (5): 1300-1307), Massively Parallel Signature Sequencing (MPSS; see e.g. Brenner et al., 2000, Nature Biotechnology, vol. 18:630-634 and Brenner et al., 2000, PNAS, vol. 97 (4):1665-1670), self-subtracted cDNA libraries (Laveder et al., 2002, Nucleic Acids Research, vol. 30(9):e38), Real-Time Multiplex Ligation-dependent Probe Amplification (RT-MLPA; see e.g. Eldering et al., 2003, vol. 31 (23): el53), High Coverage Expression Profiling (HiCEP; see e.g. Fukumura et al., 2003, Nucleic Acids Research, vol. 31(16):e94), a universal micro-array system as disclosed in Roth et al.(Roth et al., 2004, Nature Biotechnology, vol. 22 (4): 418-426), a transcriptome subtraction method (see e.g. Li et al., Nucleic Acids Research, vol. 33 (16): el36), and fragment display (see e.g. Metsis et al., 2004, Nucleic Acids Research, vol. 32 (16): el27).

The nucleic acid stabilized in the hydrogel may be used in a method for enrichment of a target nucleic acid fragment, preferably as described in PCT/EP2019/082791, which is incorporated herein by reference. Hence, the invention may pertain to a method for enrichment of a target nucleic acid fragment from a hydrogel comprising an isolated nucleic acid, wherein the target nucleic acid fragment comprises a sequence of interest, and wherein the method comprises the steps of:
i) providing a hydrogel comprising an isolated nucleic acid as defined herein, wherein the isolated nucleic acid comprises the sequence of interest;
ii) cleaving the isolated nucleic acid in the hydrogel with at least a first and a second gRNA-CAS complex, thereby generating a hydrogel comprising the target nucleic acid fragment comprising the sequence of interest and at least one non-target nucleic acid fragment;
iii) contacting the cleaved nucleic acid molecules in the hydrogel obtained in step b) with an exonuclease and allowing the exonuclease to digest the at least one non-target nucleic acid fragment in the hydrogel; and
iv) optionally purifying the target nucleic acid fragment in the hydrogel comprising the sequence of interest from the digest obtained in step c).

Step d) may additionally or alternatively comprise a step of dissolution of the hydrogel comprising the target nucleic acid fragment.

### Sequencing

In an aspect, the invention pertains to a sequencing method, in particular the invention relates to a method for sequencing an manipulated, preferably isolated, nucleic acid as defined herein.

Preferably, a sequencing library is prepared from the nucleic acid as defined herein above, i.e. the sequencing library is prepared in a hydrogel. Preferably, the sequencing library is released from the hydrogel before or during the sequencing process. Preferably, the sequencing library is released from the hydrogel before the sequencing process. Preferably, the sequencing library is released from the hydrogel by dissolving the hydrogel.

Hence, preferably the sequencing method of the invention comprises the steps of:
obtaining a sequencing library as defined herein;
dissolving the hydrogel; and
sequencing the library.
   The hydrogel is preferably dissolved at a temperature below 45° C. The lower temperature prevents, or substantially prevents, breakage of the nucleic acid. To further facilitate dissolving the hydrogel and releasing the nucleic acid, the hydrogel may have a beneficial surface area to volume ratio (SA/V ratio), e.g. the hydrogel comprises or consists of small particles to promote contact between the hydrogel and a dissolution inducer. Preferably, the hydrogel may comprise or substantially consist of small particles, preferably comprises or substantially consists of microparticles. Preferably the hydrogel comprises or substantially consists of microspheres, preferably microspheres as defined herein above.

The sequencing library may be sequenced using any conventional method known in the art for the skilled person. Preferably the sequencing is deep-sequencing, preferably long-read deep sequencing. Preferably, the sequencing is third generation sequencing. Preferably, sequencing may be performed using at least one of Roche 454A and 454B sequencing, ILLUMINA™ SOLEXA™ sequencing, Applied Biosystems' SOLID™ sequencing, the Pacific Biosciences' SMRT™ sequencing, Pollonator Polony sequencing, Oxford Nanopore Technologies or the Complete Genomics sequencing. Preferably, sequencing may be performed using at least one of nanopore sequencing and single-molecule real-time (SMRT™) sequencing. Preferably, the sequencing may comprise a step of nanopore sequencing.

Preferably, the sequencing library is released from the hydrogel before, or substantially before, the sequencing process. The sequencing library may be released from the hydrogel in the sequencer flow cell. Thus the hydrogel comprising the sequencing library may first be loaded on a sequencer flow cell, preferably wherein the hydrogel comprises or substantially consists of microparticles, preferably microspheres. In the sequencer flow cell the hydrogel may be dissolved, thereby releasing the sequencing library for subsequent sequencing, preferably long-read deep-sequencing. Preferably, the flow cell is a flow cell of a long-read deep sequencer, preferably a third generation sequencer. Preferably, the flow cell is a flow cell for single-molecule real-time (SMRT™) sequencing or a flow cell for nanopore sequencing.

Preferably, the flow cell is for nanopore sequencing.
Preferably, the sequencing library is released from the hydrogel by dissolving the hydrogel, preferably dissolving the hydrogel in the flow cell.

Preferably, the hydrogel is dissolved by the addition of a sequencing buffer, preferably a sequencing buffer suitable for nanopore sequencing.

Preferably, the hydrogel is dissolved by the addition of a buffer comprising monovalent cations, preferably potassium or sodium cations, preferably sodium.

Preferably, the hydrogel is dissolved by lowering the temperature from about 20° C.-40° C. to about 2° C.-10° C. Preferably, the nucleic acid remains in the hydrogel at a temperature that is required for an enzymatic reaction to take place. Preferably, the hydrogel does not dissolve at a temperature that is required for an efficient enzymatic reaction, preferably an enzymatic reaction for preparing a sequencing library, e.g. an enzymatic reaction at a temperature of about 37° C. Preferably the hydrogel is dissolved at a lower temperature, e.g. a temperature that is lower than the temperature for an enzymatic, preferably for an efficient enzymatic, reaction. As a non-limiting example, the hydrogel may be dissolved when placing the hydrogel at a temperature of about 4° C.

The hydrogel may be a pH-responsive hydrogel. The hydrogel may be dissolved by adjusting the pH, i.e. to decrease or increase the pH. The nucleic acid preferably remains stable at a pH of about 5-8. Hence in an embodiment, the hydrogel may be dissolved by increasing the pH, e.g. by adjusting the pH from about 5-6 to about 7-8. In an alternative embodiment, the hydrogel may be dissolved by decreasing the pH, e.g. by adjusting the pH from about 7-8 to about 5-6.

### Compositions

In an aspect, the invention relates to a composition comprising a nucleic acid as defined herein above, and a hydrogel. The nucleic acid may be stabilized within the hydrogel to render the nucleic acid less prone to breakage. The hydrogel preferably can be dissolved at a temperature below 45° C. Preferably, the hydrogel is a pH-sensitive hydrogel, preferably comprising alginate. Preferably the hydrogel comprises microparticles, preferably microspheres. Preferably, the hydrogel is a hydrogel as defined herein above.

In a further aspect, the invention pertains to a composition comprising a cell and an aqueous polymer solution as defined herein above. The invention is not limited to any specific type of cell, preferably the cell is a cell as defined herein above. Preferably the cell is a plant cell, preferably a plant protoplast.

In an aspect, the invention concerns a composition comprising an organelle and an aqueous polymer solution as defined herein above. The organelle preferably comprises a nucleic acid. The organelle is preferably an organelle as defined herein above. Preferred organelles are at least one of a nucleus, a mitochondrion and a chloroplast. A preferred organelle is a nucleus. The organelle can be obtainable from any cell, preferably a cell as defined herein above. Preferably, the organelle is obtainable from a plant cell.

### Hydrogels

In an aspect, the invention pertains to a hydrogel obtainable by the method of the invention. Preferably, the hydrogel comprises at least one of:
A nucleic acid-comprising carrier, preferably a nucleic acid-comprising carrier as defined herein;
an organelle, preferably an organelle as defined herein;
a manipulated nucleic acid, preferably an isolated nucleic acid as defined herein, preferably a μHMW nucleic acid; and
a sequencing library, preferably a sequencing library as defined herein.
   The hydrogel is preferably a hydrogel as defined herein. The hydrogel preferably comprises alginate.

### Kit of Parts

In an aspect, the invention pertains to a kit of parts, preferably for use in method as defined herein.

The kit of parts may comprise a polymer for forming a hydrogel as defined herein. The kit of parts may further comprise at least one of
a lysis buffer for lysing at least one of a cell and an organelle; and
one or more components for preparing a sequencing library, preferably a deep-sequencing library.

The polymer may be an aqueous solution. Alternatively, the polymer is in lyophilized form and can be reconstituted with an aqueous solution for use in the method of the invention.

The lysis buffer for lysing at least one of a cell and an organelle may constitute any lysis buffer known in the art that is suitable for cell and/or organelle lysis. Preferably the lysis buffer does not, or does not substantially, destroy the formation of the hydrogel. Non-limiting examples of a cell lysis buffer are NP-40 lysis buffer, RIPA lysis buffer, SDS lysis buffer and ACK lysis buffer.

The one or more components for preparing a sequencing library may comprise at least one of an enzyme and an adapter. The enzyme may be an ligase. The adapter may be an adapter as defined herein above.

Alternatively or in addition, the kit of part may comprise a hydrogel, preferably a hydrogel as defined herein. The hydrogel may comprise at least one of a nucleic acid, nucleic acid-comprising carrier, a cell, an organelle and an isolated nucleic acid, preferably at least one of a nucleic acid-comprising carrier, a cell, an organelle and an isolated nucleic acid as defined herein. The kit may further comprise one or more components for preparing a sequencing library, preferably a deep-sequencing library. The one or more components for preparing a sequencing library may comprise at least one of an enzyme and an adapter. The enzyme may be an ligase. The adapter may be an adapter as defined herein above.

Preferably, the volume of any of the vials within the kit do not exceed 100 mL, 50 mL, 20 mL, 10 mL, 5 mL, 4 mL, 3 mL, 2 mL or 1 mL.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

### Further Aspects

In an aspect, the invention concerns the use of a composition as defined herein above. It is clear for the skilled person that the invention is not limited to (deep-)sequencing of the isolated and stabilized nucleic acid as defined herein. A stabilized nucleic acid as defined herein can find numerous applications.

Preferably, the invention pertains to a use of composition as defined herein for at least one of genome sequencing, long-range genome analysis, transcriptome analysis, map-based cloning, genome physical mapping, the construction of a large-insert BAC library, and the construction of a large insert BIBAC library.

Similarly in further aspects, the invention relates to at least one of a method for genome sequencing, a method for long-range genome analysis, a method for transcriptome analysis, a method for map-based cloning, a method for genome physical mapping, a method for the construction of a large-insert BAC library, and a method for the construction of a large insert BIBAC library, wherein the method preferably comprises the steps of:
a) combining a carrier, comprising the nucleic acid, preferably at least one of an organelle and a cell, with an aqueous polymer solution;
b) gelling the polymer solution to form a hydrogel comprising at least one of the organelle and the cell; and
c) releasing the nucleic acid from the carrier to obtain the isolated nucleic acid,
wherein the isolated nucleic acid is stabilized in the hydrogel.

Preferably, the nucleic acid is a nucleic acid as defined herein above. Preferably, the hydrogel is a hydrogel as defined herein above.

### FIGURE LEGEND

FIG. 1. Exemplary embodiments of the invention. (FIG. 1A) Embodiment depicting encapsulation of nuclei in hydrogel beads followed by nuclei lysis and purification of the nucleic acids after gelling of the hydrogel. Step 1) Nucleic acids in a biological carrier, such as cells or nuclei, 2) Encapsulation of the biological carrier in a hydrogel, 3) Reversible gelling of the hydrogel and lysis of the biological carrier, 4) Purification and manipulation (e.g. sequence library preparation) of nucleic acids, 5) De-gelling and recovery of nucleic acids, 6) Further processing of nucleic acids (e.g. sequencing) (FIG. 1B) In another exemplary embodiment, nucleic acids (1) can be directly encapsulated in the hydrogel (2) followed by reversible gelling of the polymer and manipulation of the nucleic acids (3). Step 4) is the de-gelling and recovery of nucleic acids and 5) further processing of the nucleic acids. (FIG. 1C) Nucleic acids may also be associated in or to a carrier other than cells or organelles, for example artificial beads or scaffolds. Step 1) Presence of nucleic acids in or to a carrier other than biological cells or organelles, 2) encapsulation of nucleic acid-carrier complex in a hydrogel, 3) reversible gelling of the hydrogel and manipulation of the nucleic acids, 4) de-gelling and recovery of nucleic acids and 5) further processing of nucleic acids. In the shown exemplary embodiments, the trapped nucleic acids can be semi-solid state-based (enzymatically) manipulated (for example, library preparation) followed by further processing upon or after de-gelling of the hydrogel. Microspheres containing the sequence library can be “loaded” directly in the flow cell followed by dissolving the particles. The released library DNA molecules become accessible to the sequencing process

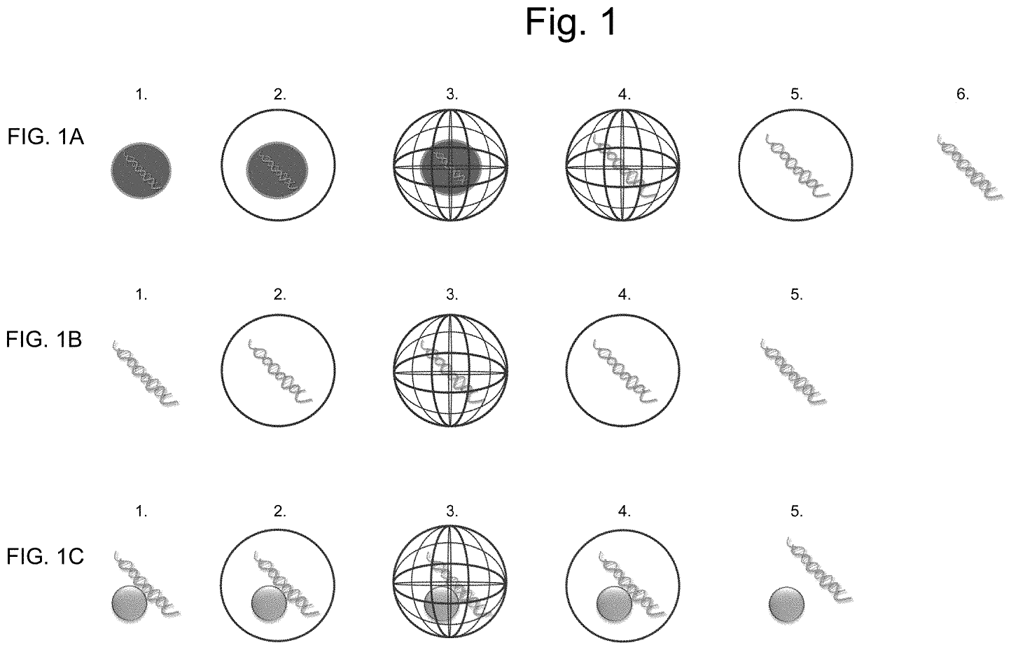

FIG. 2. Percentage of genomic DNA loss by diffusion from the hydrogel beads in the different buffers used during semi-solid state Nanopore library preparation of Example 1 and 2. Indicated is the amount of relative DNA recovered from each discarded buffer used during Nanopore library preparation incubation, i.e. buffer solution used for washing prior to DNA repair and end-preparation (1), buffer comprising enzymes for DNA repair and end preparation (2), MQ for washing after DNA repair and end preparation (3), ligation buffer for washing prior to ligation (4), ligation buffer comprising ligase and adapters for adapter ligation (5) and elution buffer for equilibration (6). The amount of input DNA used for encapsulation is set at 100%. In all fractions and for all Examples, almost no DNA was lost in the aqueous solutions indicating no or a very low level of diffusion of encapsulated DNA from the hydrogel in the solution throughout library preparation, with the highest percentage of loss during the step of adapter ligation (see, inset graph), however, size determination showed that this was unligated adapter DNA (data not shown).

FIG. 3. Read length distribution of all reads that mapped against a reference genome. On the x-axis, all individual reads that map against the reference are given. The y-axis shows the read length in bases.

FIG. 4. Schematic presentation of microfluidic-based synthesis of hydrogel microspheres comprising nucleic acid-comprising carriers, wherein the first reactant comprises nucleic acid-comprising carriers and the second reactant comprises the aqueous polymer solution. An optional third reactant may comprise a gelation inducer. 1A) Dispersed phase, e.g. cells, organelles, nucleic acids, biomolecules, 1B) Dispersed phase, aqueous polymer solution, 2) Continuous phase, e.g. oil-surfactant emulsion, 3) Collection phase.

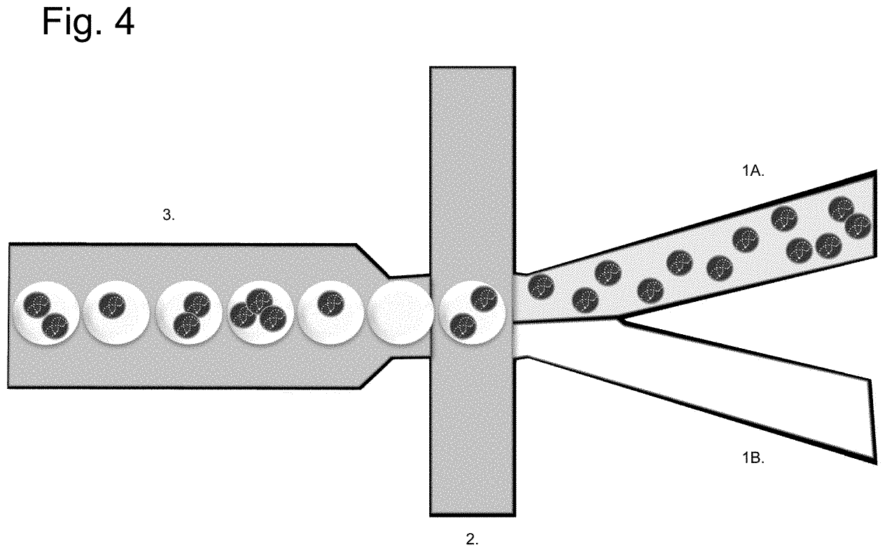

FIG. 5. Pulse-field gel electrophoresis of uHMW genomic plant DNA. FIG. 5A.) Lane 1: Nuclear DNA isolated after lysis of sugar beet nuclei and further purification in an aqueous environment. Lane 2: Saccharomyces cerevisiae chromosomal pulse-field marker with the size of the chromosomes given in kb. Molecular weights of individual fragments are indicated next to the gels. FIG. 5B.) Lane 1: Saccharomyces cerevisiae chromosomal pulse-field marker. Lanes 2, 4 and 5: Genomic DNA derived from sugar beet, nuclei that were encapsulated and lysed in alginate spheres. Lane 3: Genomic DNA derived from Impatiens, nuclei that were encapsulated and lysed in alginate spheres. In lane 2 and 3, alginate spheres containing uHMW DNA were loaded directly in the pulse-field gel prior electrophoresis. In lane 4 and 5, the uHMW DNA was released from the alginate spheres by addition of sodium citrate to the wells.

### Examples

Example 1a and b: Encapsulation of high molecular weight bacteriophage Lambda DNA in alginate, followed by semi-solid state library preparation and Nanopore sequencing

### Example 1a

10 μL Escherichia virus Lambda (λ) DNA (total amount of DNA: 883 ng) with a median size of 45 Kb and a concentration of 82.4 ng/μL was mixed overnight with 10 μL 1.5% Pronova® UP LVG alginate (NovaMatrix™) on a rotary platform at 4° C. After incubation, the alginate solution was pipetted into a 2 ml BD Plastipak™ syringe (BD) using a 200 μL wide bore pipette tip to avoid shearing of the DNA. An 0.45×13 mm microlance (BD) was attached to the syringe and the alginate solution was slowly dripped into a 100 mL beaker glass containing 20 mL of a 200 mM CaCl₂ solution. To obtain round-shaped, millimetre-sized beads, the CaCl₂ solution was stirred vigorously by placing the beaker glass with a magnetic rod on a magnetic stirrer plate prior dripping of the alginate solution. With this dripping method, about one to two beads could be generated using 20 μL 0.7% alginate-DNA mixture.

After gelling, the beads containing the encapsulated A DNA were transferred to a 2-mL Eppendorf tube and were incubated for 30 minutes in 60 μL DNA repair and end-preparation mixture (without enzymes) containing 17.1× diluted NEBNext® FFPE repair and Ultra II End-prep reaction buffers (NEBNext® Companion Module for Oxford Nanopore Technologies® Ligation Sequencing, New England Biolabs Inc.) for washing prior to DNA repair and end-preparation. The NEBNext® buffer solution was discarded by pipetting.

Subsequently, for DNA-repair and end preparation, the beads were incubated for 30 minutes at 20° C. and 10 minutes at 65° C. in 60 μL NEBNext® FFPE repair and Ultra II End-prep reaction mixture containing 17.1× diluted NEBNext® FFPE repair and Ultra II End-prep reaction buffers, 30× diluted NEBNext® FFPE DNA Repair Mix, and 20× diluted NEBNext® Ultra II End-prep Enzyme Mix (NEBNext® Companion Module for Oxford Nanopore Technologies® Ligation Sequencing, New England Biolabs Inc.).

After DNA repair and end preparation, the beads were washed in 500 μL nuclease-free water followed by a 30 minutes incubation at 20° C. in a fresh batch of 500 μL nuclease-free water. The water was discarded by pipetting and the beads were incubated for 30 minutes in 100 μL adapter ligation mixture (without enzymes and without adapters) containing 4× diluted Ligation Buffer (SQK-LSK109 Ligation Sequencing Kit, Oxford Nanopore Technologies) prior to adapter ligation. The ligation buffer solution was discarded by pipetting and the beads were incubated for 2 hours at 4° C. in 100 μL adapter ligation mixture containing 4× diluted Ligation Buffer (SQK-LSK109 Ligation Sequencing Kit, Oxford Nanopore Technologies), 10× diluted NEBNext® Quick T4 DNA Ligase (NEBNext® Companion Module for Oxford Nanopore Technologies Ligation Sequencing, New England Biolabs Inc.) and 20× diluted Adapter Mix (SQK-LSK109 Ligation Sequencing Kit, Oxford Nanopore Technologies) for adapter ligation.

After adapter ligation, the beads were equilibrated for 30 minutes at room temperature in 50 μL Elution Buffer (SQK-LSK109 Ligation Sequencing Kit, Oxford Nanopore Technologies). Meanwhile, a R9.4.1 flow cell (Oxford Nanopore Technologies) was primed according the manufacturer's instructions (Nanopore protocol Genomic DNA by Ligation, version GDE_9063_v109_revN_14Aug2019, Oxford Nanopore Technologies). Following equilibration of the beads, the Elution Buffer was discarded and the beads were incubated for 30 minutes on ice in 75 μL 2× diluted Sequencing Buffer (SQK-LSK109 Ligation Sequencing Kit, Oxford Nanopore Technologies).

Incubation of the alginate beads in Sequencing Buffer caused de-gelling of the alginate bead. The alginate slurry containing the λ DNA 1D sequencing library was loaded via the SpotON sample port in the R9.4.1 flow cell (FAL16833) and the sequencing run was started on a GridION sequencer (MinKNOW version 3.4.8). The raw sequence data was base called with Guppy version 3.0.6 (Oxford Nanopore Technologies®) and the base called sequence data was further processed with NanoPack tools (De Coster et al., 2018. NanoPack: visualizing and processing long-read sequencing data. Bioinformatics 34-15).

### Example 1b

In Example 1 b, the semi-solid state library was prepared as described above, with the exception that 200 mM sodium citrate was added to the sequencing buffer, i.e. in the last step, the beads were incubated in 75 μL 2× diluted Sequencing Buffer (SQK-LSK109 Ligation Sequencing Kit, Oxford Nanopore Technologies) and 200 mM sodium citrate. The de-gelled alginate slurry with the A DNA library was loaded in a new R9.4.1 flow cell (FAL02990) and the sequencing was performed on a GridION sequencer.

Summary statistics of the two 42 hours λ DNA sequence runs are presented in Table 1.

TABLE 1
NanoPlot analysis of MinION sequencing data.

| | Example 1a | Example 1b |
|---|---|---|
| Mean read length (bp) | 13,654.4 | 8,128.9 |
| Mean read quality | 7.4 | 7 |
| Median read length (bp) | 3,292 | 702 |
| Median read quality | 7.3 | 3.8 |
| Number of reads | 97,693 | 39,999 |
| Read length N50 (bp) | 45,303 | 34,117 |
| Total bases | 1,333,941,793 | 325,149,673 |

Sequencing of the semi-solid state prepared Example 1a λ DNA library has produced more than one gigabase of data represented by about 98,000 reads. The median read length is 3.3 Kb with a median read quality of 7.3. In contrast, the sequence yield of the λ DNA sequence run of Example 1b is more than fourfold lower (about 325 megabases) and median read length is even smaller than 1 kb (702 bases). The differences in sequence metrics between both examples is due to the differences in the post-library preparation treatment of the large beads; i.e. the de-gelling and loading of the alginate in sequence buffer (Example 1a) or sequence buffer with 200 mM sodium citrate added (Example 1b).

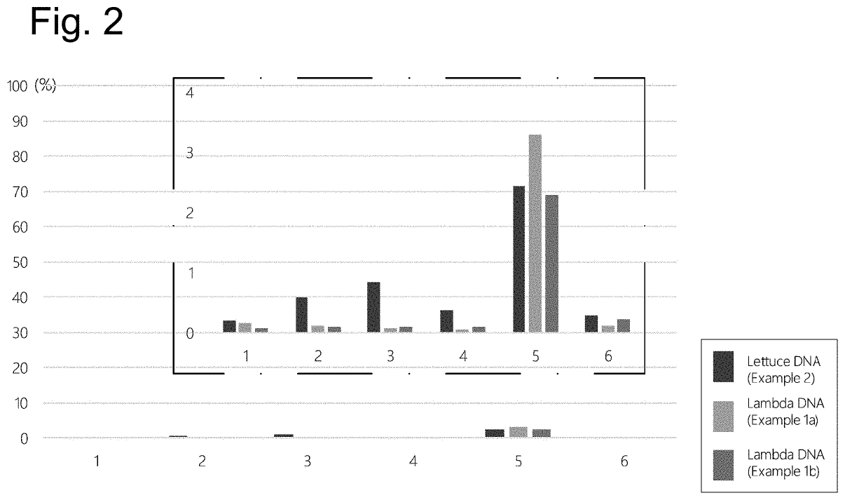

In order to investigate that the lambda sequence reads originated from entrapped DNA and not from DNA that has been diffused out the beads into the aqueous solutions during the library preparation steps, DNA quantity was measured after each incubation step. FIG. 2 shows the relative DNA loss by diffusion in the different washing and enzymatic steps during library preparation. DNA was isolated from the used buffers and enzyme mixes with Ampure XP beads and the quantity was determined with a Qubit fluorometer (High Sensitive dsDNA Assay kit, ThermoFisher Scientific). As is shown in FIG. 2, no DNA was detectable in the different aqueous solutions throughout the entire Nanopore library preparation procedure. After incubation in the ligation enzyme mixture, containing also the sequence adapters, a low amount of low molecular weight (˜200 bp) DNA could be observed. However, based on the size of the fragments and the composition of the ligation mixture, we conclude that the recovered DNA are non-ligated Nanopore adapters.

In summary, the results show that the present invention provides for a useful platform for preparation of long read sequence libraries starting from encapsulated HMW DNA in semi-solid state.

### Example 2: Encapsulation of High Molecular Weight Plant DNA in Alginate, Followed by Semi-Solid State Library Preparation and Nanopore Sequencing

For example 2, the same experimental procedure was followed as described for example 1a, with the difference of mixing 993 ng (10 μL) genomic plant DNA with a median peak size of about 80 kb and a size range of 10 to 128 kb with the alginate. The alginate slurry containing the plant DNA 1D sequencing library was loaded via the SpotON sample port in the R9.4.1 flow cell (FAK94960). Sequencing was performed on a GridION (MinKNOW version 3.4.8) and the raw sequence data was base called with Guppy version 3.0.6 (Oxford Nanopore Technologies®). Further analyses of the base called sequence data was done with NanoPack tools (De Coster et al., 2018. NanoPack: visualizing and processing long-read sequencing data. Bioinformatics 34-15).

Summary statistics of the 42 hours sequence run are presented in Table 2.

TABLE 2
NanoPlot analysis of MinION sequencing data

| | Example 2 |
|---|---|
| Mean read length (bp) | 4,025 |
| Mean read quality | 4.7 |
| Median read length (bp) | 577 |
| Median read quality | 3.4 |
| Number of reads | 55,165 |
| Read length N50 (bp) | 26,126 |
| Total bases | 222,040,158 |

Sequencing of the semi-solid state prepared plant DNA library resulted in slightly more than 222 Mb of data represented by 55,165 reads. These results demonstrate the possibility for preparation and sequencing of long read sequence libraries starting from encapsulated ultra HMW DNA in semi-solid state.

Quantification of the amount of lettuce DNA in the different aqueous solutions during library preparation showed that, like for the lambda DNA examples, no diffusion has occurred during incubation of the hydrogel beads. Therefore, we conclude that the sequence reads obtained were derived from encapsulated DNA and were generated by means of semi-solid state library preparation. As was the case for the lambda Example, the small amount of DNA that was recovered in step 5 was adapter DNA.

### Example 3: Encapsulation and Lysis of Plant Nuclei in Alginate, Followed by Semi-Solid State Library Preparation and Nanopore Sequencing of the Embedded DNA

Nuclei were isolated from young leaf tissue essentially following the instructions described for plant, algal or fungal tissues in Zhang et al., 2012 (Preparation of megabase-sized DNA from a variety of organisms using the nuclei method for advanced genomics research, Nature Protocols, vol. 7 (3): 467-478). After the final wash step, the nuclei were resuspended in a 1% alginate (A0682 alginic acid sodium salt from brown algae, Sigma-Aldrich) solution containing 0.5× phosphate buffered saline (PBS; 69 mM NaCl, 5 mM phosphate, 1.4 mM KCl, pH 7.4), 1 mM CaCl₂, and 5 mM MgCl₂.

After complete resuspension of the nuclei, the suspension was pipetted into a 2 ml BD Plastipak™ syringe (BD) with an 0.45×13 mm microlance (BD) attached using a 200 μL wide bore pipette tip to avoid damage of the nuclei. Subsequently, the alginate solution was slowly dripped into a 100 mL beaker glass containing 20 mL of a 200 mM CaCl₂ solution under stirring conditions. The resulting millimetre-sized alginate beads containing plant nuclei were further incubated for 30 minutes in the 200 mM CaCl₂ solution without stirring to facilitate further gelling of the alginate. After incubation of the bead in propidium iodide, a large number of fluorescent nuclei could be observed.

After complete gelling of the alginate, the beads were collected using a 500 μm Pluristrainer (PluriSelect Life Science) and transferred to a new 50 mL polypropylene tube containing 20 mL of lysis solution containing TE buffer (10 mM Tris-HCl and 1 mM EDTA, pH 8.0), 50 mM CaCl₂ and 300 μg/mL proteinase K and the nuclei were lysed overnight at 50° C. in a shaking incubator with an orbital agitation set at 75 rpm.

Following lysis, the beads containing the entrapped genomic DNA were washed twice for 30 minutes in fresh 20 mL wash solutions (TE buffer and 50 mM CaCl₂). Complete deactivation of proteinase K was established by overnight incubation at 37° C. in a 20 mL solution containing TE buffer and 2 mM Pefabloc® SC (Sigma-Aldrich). The Pefabloc® SC was removed by incubating the beads twice in 20 mL TE buffer and 50 mM CaCl₂ solution and the beads containing the encapsulated genomic DNA were stored in the same solution at 4° C. until use.

For nanopore library preparation, 4 beads were incubated for 30 minutes in 30 μL DNA repair and end-preparation mixture containing 8.6× diluted NEBNext® Ultra II End-prep reaction buffer (NEBNext® Ultra™ II End Repair/dA-Tailing Module, New England Biolabs Inc.), 0.83× NAD+ (PreCR® Repair Mix, New England Biolabs Inc.) and 5 mM CaCl₂. The solution was discarded by pipetting and the beads were incubated for one hour at 20° C. and 20 minutes at 65° C. in 60 μL NEBNext® FFPE repair and Ultra II End-prep reaction mixture containing 8.6× diluted NEBNext® Ultra II End-prep reaction buffer (NEBNext® Ultra™ II End Repair/dA-Tailing Module, New England Biolabs Inc.), 0.83× NAD+ (PreCR® Repair Mix, New England Biolabs Inc.), 5 mM CaCl₂, 20× diluted NEBNext® FFPE DNA Repair Mix (New England Biolabs Inc.), and 1.3× diluted NEBNext® Ultra II End-prep Enzyme Mix (NEBNext® Ultra™ II End Repair/dA-Tailing Module, New England Biolabs Inc.).

Following DNA repair and end preparation, the beads were washed with 1 mL 10 mM Tris-HCl and 5 mM CaCl₂ solution and subsequently incubated for 15 minutes incubation at 20° C. in a fresh batch of 10 mM Tris-HCl and 5 mM CaCl₂ solution. This incubation step was repeated once. After the second incubation step, the solution was discarded and the beads were incubated for 30 minutes at 20° C. in 50 μL adapter ligation mixture containing 2× diluted NEBNext® Ultra II Ligation Master Mix (NEBNext® Ultra™ II Ligation Module, New England Biolabs Inc.), 50× diluted NEBNext® Ligation Enhancer (NEBNext® Ultra™ II Ligation Module, New England Biolabs Inc.), 10 μL Oxford Nanopore Technologies Adaptor Mix (SQK-LSK108 Ligation Sequencing Kit 1D, Oxford Nanopore Technologies), and 5 mM CaCl₂. After incubation, the ligation mixture was discarded and the ligation step was repeated with a fresh ligation mixture.

Following adapter ligation, the beads were equilibrated for two times 30 minutes at room temperature in 25 μL and 50 μL Elution Buffer (SQK-LSK108 Ligation Sequencing Kit 1D, Oxford Nanopore Technologies), respectively. Meanwhile, a R9.4 flow cell (ID FAH05684, Oxford Nanopore Technologies) was primed according the manufacturer's instructions (1D gDNA selecting for long reads, SQK-LSK108, Oxford Nanopore Technologies). After the equilibrations in the elution buffers, the beads were incubated for 30 minutes at 20° C. in 75 μL pre-sequencing mix containing 35 μL Running Buffer Fuel (RBF; SQK-LSK108 Ligation Sequencing Kit 1D, Oxford Nanopore Technologies), 4 μL Elution Buffer (SQK-LSK108 Ligation Sequencing Kit 1D, Oxford Nanopore Technologies), 25.5 μL Library Loading Beads (EXP-LLB001 Library Loading Beads Kit, Oxford Nanopore Technologies), and 10 μL 1.5 M sodium citrate. The beads were incubated for 30 minutes at 20° C. and the slurry was loaded using a wide-bore pipet via the SpotON sample port in the R9.4 flow cell and the sequencing run was started on a MinION MK 1 b sequencer.

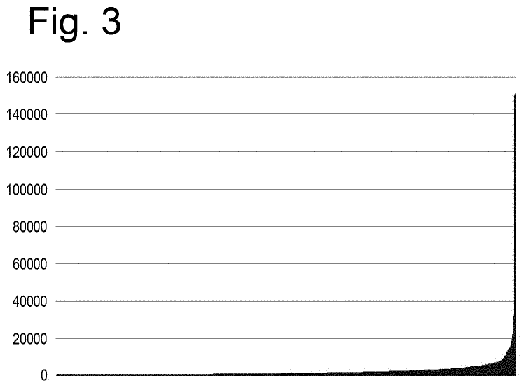

Sequencing of the semi-solid state prepared plant DNA library resulted in 1,448 reads that could be mapped (Minimap2) against a reference whole genome sequence (FIG. 3). More than ten percent of the reads mapped with a similarity equal to or greater than 90%. These results show the possibility for preparation and sequencing of long read sequence libraries in a semi-solid state fashion starting from encapsulated plant nuclei.

### Example 4: Effect of Encapsulation and Lysis of Plant Nuclei in Alginate on DNA Size

The effect of protecting nuclear DNA in alginate spheres was further analysed. To this end, genomic DNA was obtained after lysis of sugar beet nuclei and further purification in an aqueous environment. The fragment size of this conventionally isolated DNA was compared with the fragment size of genomic DNA isolated from alginate-embedded nuclei.

For the alginate-embedded samples, sugar beet and impatiens nuclei were encapsulated and lysed in alginate spheres according to the procedure described in Example 3. The alginate-embedded μHMW DNA was either loaded directly on a gel, or was first released from the alginate spheres and subsequently loaded on a gel.

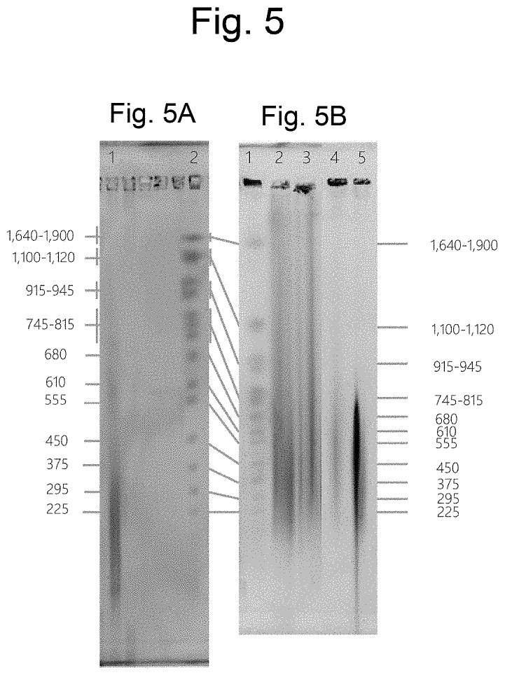

As shown in FIG. 5, a gentle isolation procedure in solution typically results in genomic DNA with a size range between 50 to 300 kb. In stark contrast, lysing sugar beet or impatiens nuclei in alginate spheres results in DNA ranges between 200 kb and megabase-size with the majority of fragments between 200 and 800 kb. Lysing the nuclei in alginate thus significantly protects the genomic DNA from break down.

1. A method for obtaining a hydrogel comprising a long manipulated nucleic acid, the method comprising:
(a) combining a nucleic acid with an aqueous polymer solution;
(b) gelling the polymer solution to form a hydrogel comprising the nucleic acid; and
(c) manipulating the nucleic acid in the hydrogel,
wherein the hydrogel can be dissolved at a temperature below 45° C.

2. The method according to claim 1, wherein the nucleic acid is provided in a carrier.

3. The method according to claim 2, wherein the carrier is at least one of an organelle and a cell.

4. The method according to claim 3, further comprising (c) lysing the organelle or to release the nucleic acid.

5. The method according to claim 1, wherein the polymers in the aqueous solution are at least one of:
(i) ionic polymers, preferably anionic polymers having carboxylic pendant groups; and
(ii) polysaccharides or derivatives thereof.

6. The method according to claim 5, wherein the polysaccharides or derivatives thereof comprise uronic acid.

7. The method according to claim 5, wherein the polysaccharides or the derivatives thereof are an alginate or a derivative thereof.

8. The method according to claim 2, wherein the carrier is a mitochondrion, a chloroplast, a nucleus, a plant cell, or a protoplast.

9. The method according to claim 1, wherein the nucleic acid is a DNA molecule.

10. The method according to claim 1, wherein the long manipulated nucleic acid is an isolated ultra-high molecular weight (uHMW) nucleic acid

11. The method according to claim 1, wherein the long manipulated nucleic acid is stabilized in a hydrogel microsphere.

12. A method for preparing a sequencing library, comprising:
(a) obtaining a hydrogel comprising a long manipulated nucleic acid according to claim 1; and
(b) modifying the nucleic acid in the hydrogel to obtain a sequencing library.

13. A sequencing method, comprising:
(a) obtaining a sequencing library according to claim 12;
(b) dissolving the hydrogel, preferably at a temperature below 45° C.; and
(c) sequencing the library.

14. The method according to claim 13, wherein the hydrogel is dissolved at a temperature below 45° C.

15. The method according to claim 14, wherein the sequencing library is loaded on a sequencer flow cell before dissolving the hydrogel.

16. The method according to claim 13, wherein the hydrogel is dissolved by at least one of:
(i) addition of a sequencing buffer;
(ii) addition of a buffer comprising monovalent cations;
(iii) lowering the temperature from about 20° C.-40° C. to about 2° C.-10° C.; and
(iv) adjusting the pH from about 5-6 to about 7-8, or from about 7-8 to about 5-6.

17. The method according to claim 16, wherein the monovalent cations are sodium cations.

18. A method for obtaining a hydrogel comprising a nucleic acid-comprising carrier, the method comprising:
(a) combining the nucleic acid-comprising carrier with an aqueous polymer solution; and
(b) gelling the polymer solution to form the hydrogel comprising the nucleic acid-comprising carrier,
wherein the hydrogel is a hydrogel according to claim 1.

19. The method according to claim 18, wherein the nucleic acid-comprising carrier is an organelle and wherein the method further comprises lysing a cell to obtain the organelle.

20. A hydrogel comprising at least one of a nucleic acid-comprising carrier, and an organelle and a long manipulated nucleic acid, wherein the hydrogel can be dissolved at a temperature below 45° C.

21. A kit of parts for obtaining a hydrogel comprising a long manipulated nucleic acid, wherein the kit comprises:
(i) a polymer for forming a hydrogel according to claim 20;
(ii) a cell and/or organelle lysis buffer; and
(iii) optionally, one or more components for preparing a sequencing library.
